(19) United States
(12) Patent Application Publication
(10) Pub. No.: US 2021/0038259 A1
(43) Pub. Date: Feb. 11, 2021

(54) PERINEAL HEATING DEVICE

(71) Applicant: UNIVERSITY OF UTAH RESEARCH FOUNDATION, Salt Lake City, UT (US)

(72) Inventors: John LANGELL, Sandy, UT (US); Alli STRAUBHAR, Salt Lake City, UT (US); Karissa DeAnn KEENAN, Salt Lake City, UT (US); John LACKEY, Sandy, UT (US); Joshua N. BURTON, West Valley City, UT (US); Timothy PICKETT, Highland, UT (US)

(21) Appl. No.: 16/981,277

(22) PCT Filed: Mar. 29, 2019
(86) PCT No.: PCT/US2019/024805

§ 371 Date: Sep. 15, 2020

Related U.S. Application Data
(60) Provisional application No. 62/651,482, filed on Apr. 2, 2018.

(51) Int. Cl. A61B 17/42; A61F 7/00; A61F 13/02

(52) U.S. Cl. CPC A61B 17/42; A61F 7/007; A61F 13/02; A61F 2007/0226; A61F 2007/005; A61F 2007/0078; A61F 2007/0071

(57) ABSTRACT

A perineal heating pad includes a central portion and two opposing end portions. The central portion includes a heating element disposed on or within the heating pad and the opposing ends may include one or more tabs extending outward from the central portion. A non-stick adhesive is used to secure the heating pad to the perineal body tissue of the patient and a controller supplies heat to the heating element and maintains the perineal tissue of a patient at a desired temperature.

### CROSS-REFERENCE TO RELATED APPLICATIONS

This Application claims priority to and the benefit of U.S. Provisional Patent Application Ser. No. 62/651,482, filed on Apr. 2, 2018 and titled “Intra-Parineal Heating Device,” the entirety of which is incorporated herein by this reference.

### BACKGROUND OF THE INVENTION

### 1. The Field of the Invention

The present disclosure relates generally to systems, methods, and apparatus for heating tissue. More specifically, the present disclosure relates to systems, methods, and apparatus for heating perineal body tissue during labor and delivery.

### 2. Background and Relevant Art

There are approximately 2.7 million vaginal deliveries in the US annually. During a vaginal delivery, there is a risk to the birth canal ranging from small lacerations to severe lacerations with long term effects. Approximately 75-85% of women who undergo a vaginal delivery will have a laceration and 4% of those will be significant injuries (3rd or 4th degree laceration of the perineal body). Large lacerations often require extensive repair with an increased risk of postpartum complications, including hemorrhage, infection & increased postpartum pain.

There are also long-term sequelae for mother's including incontinence of fecal material, chronic pain, increased risk of pelvic organ prolapse and the need for surgical treatment later in life. Obstetric providers and patients are interested in ways to prevent severe perineal lacerations and avoid narcotic use, which is often given for postpartum pain management.

Currently, methods and devices for preventing perineal lacerations during labor and delivery include manual application of warm compresses to the perineum. Such warm compresses include a cloth soaked in warm water, which is held against the perineal tissue during labor and delivery. This results in unpredictable, cyclical temperatures being applied to the perineal tissue as the water cools during use and newly soaked cloths are reapplied. In addition, holding a warm compress or cloth against the perineal tissue requires one or more extra hands that may get in the way of other medical professionals performing other urgent care to the same area of the patient during labor and delivery.

Other typical warm compress devices are also not conducive for use at the perineal body tissue area of a patient during labor and delivery. For example, warm compress devices often lack precise and consistent temperature control. Current heating pads and other devices are also not effectively designed for the physical contours and features of the perineal tissue and surrounding anatomy of a patient and/or for use in a wet environment such as encountered during labor and delivery. In addition, such devices may impede the use of other medical devices used during labor and delivery or block the vaginal opening or anus of the patient.

Accordingly, there are a number of problems in the prior art that need to be addressed.

### BRIEF SUMMARY

Implementations of the present disclosure solve one or more problems in the art with systems, methods, and apparatus for heating perineal body tissue of a patient during labor and delivery. In at least one embodiment, a device for heating perineal body tissue during vaginal labor and deliver includes a laminar pad having a central portion and first and second end portions. The central portion comprises a heating element. The first end portion and the second end portion each extend from the central portion, the central portion being narrower than each of the first and second end portions. In addition, the laminar pad includes a backing layer and an adhesive layer.

In at least one embodiment, a system for heating perineal body tissue includes a heating pad and a battery. The heating pad includes a water-proof backing layer and a non-stick adhesive layer secured to the backing layer. A heating element is disposed between the backing layer and the adhesive layer in the central portion of the heating pad. The battery is connected to the heating element and provides electrical current to the heating element to heat the heating element through resistive heating.

In at least one embodiment, a method for heating perineal body tissue of a patient during labor and delivery includes providing a heating pad. The heating pad includes a central portion disposed between two opposing end portions. The heating pad also includes a non-stick adhesive layer, a water-proof backing layer secured to the adhesive layer, and a heating element disposed between the adhesive layer and the backing layer in the central portion. The method also includes applying the adhesive layer to contact the perineal tissue of a patient and securing the end portions of the heating pad to tissue surrounding the perineal tissue. Also, the method includes providing electrical current to the heating element.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A illustrates a first-degree perineal laceration;

FIG. 1B illustrates a second-degree perineal laceration;

FIG. 1C illustrates a third-degree perineal laceration;

FIG. 1D illustrates a fourth-degree perineal laceration;

FIG. 2A illustrates a perspective view of an embodiment of an intra-perineal heating device;

FIG. 2B illustrates a top view thereof;

FIG. 3 illustrates an embodiment of an intra-perineal heating device in use on the perineal tissue of a patient;

FIG. 4A illustrates a top view of an embodiment of an intra-perineal heating device;

FIG. 4B illustrates a top view of an embodiment of an intra-perineal heating device;

FIG. 4C illustrates a top view of an embodiment of an intra-perineal heating device;

FIG. 4D illustrates a top view of an embodiment of an intra-perineal heating device;

FIG. 5 illustrates an exploded view of an embodiment of an intra-perineal heating device including a backing layer, a heating element, and an adhesive layer;

FIG. 6A illustrates a perspective view of an embodiment of an intra-perineal heating device;

FIG. 6B illustrates a perspective view of an embodiment of an intra-perineal heating device;

FIG. 7 illustrates an embodiment of an intra-perineal heating device having a heating element connected to a temperature controller; and

FIG. 8 illustrates a flowchart describing an embodiment of a method of preventing perineal tissue lacerations during labor and delivery.

### DETAILED DESCRIPTION OF THE PREFERRED EMBODIMENTS

Implementations of the present disclosure solve one or more problems in the art with systems, methods, and apparatus for heating perineal body tissue of a patient during labor and delivery. In at least one embodiment, an intra-perineal heating device includes a heating pad and a heating element disposed on or within the heating pad. In addition, the device includes a non-stick adhesive disposed on a side of the heating pad that contacts the perineal body tissue of the patient. In at least one implementation, the heating element is electrically connected to a power source and/or a digital controller that maintains the heating pad at a desired temperature or provides a desired temperature profile over time.

The intra-perineal heating device of the present disclosure safely provides heat to the perineal body tissue of a patient and adheres to the tissue even under wet conditions. The heating device is water-proof and comfortable for the patient to wear. Also, the heating device can be easily applied to the patient without interfering with delivery or postpartum care, and removal of the pad does not result in residue or the removal of hair from the patient.

In addition, the temperature of the heating device is easily regulated and controlled to maintain ideal temperatures that reduce the risk of perineal lacerations forming during labor and delivery. As a result, embodiments of the heating device described herein reduce pain during labor and delivery as well as postpartum pain, thus reducing the need for narcotic pain management.

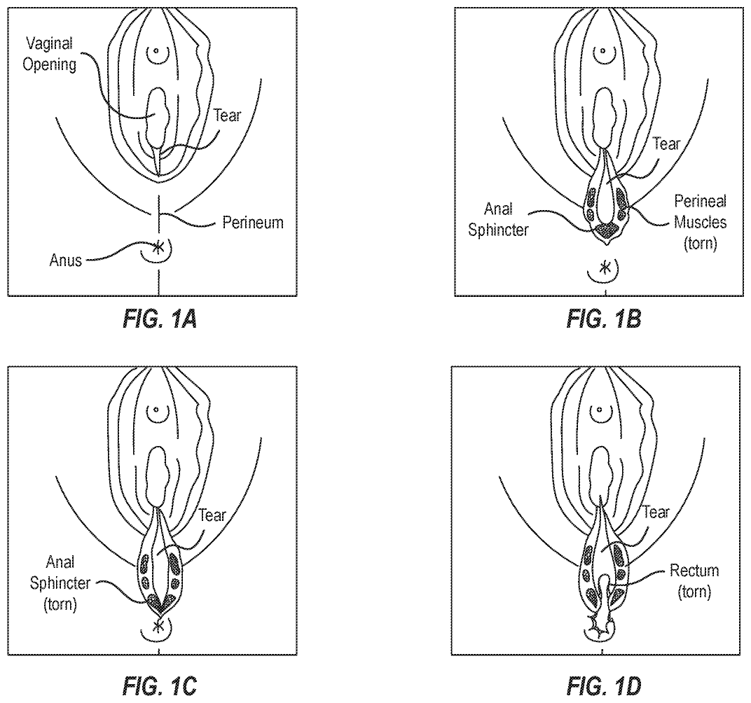

Turning now to the figures, FIGS. 1A-1D illustrate varying degrees of perineal lacerations (or “tears”) that may form in the perineal body tissue of a patient during labor and delivery. The perineal tissue of the patient extends between the vaginal opening and anus of the patient and undergoes strain during labor and delivery as the cervix dilates and stresses surrounding tissue. This stress can cause tearing of perineal body tissue, including underlying perineal muscles and the anal sphincter to tear. In the most sever cases, the perineal tear extends through the rectum.

As generally understood, perineal tears are classified into four degrees. FIG. 1A illustrates a first-degree perineal tear. First-degree perineal tears are the least severe and may or may not require stitches but still cause pain during and after labor and delivery. First degree perineal tears are generally limited to the tearing of the vaginal tissues and do not extend to perineal muscle tissue between the vaginal opening and the anus.

FIG. 1B illustrates a second-degree perineal tear. Second-degree perineal tears are more severe and involve tearing of the perineal muscles. These muscles support the bladder, vagina, and uterus and require stitches to repair the perineum. Second-degree perineal tears may cause the anal sphincter to be exposed but does not result in a torn anal sphincter.

As shown in FIG. 1C, a third-degree perineal tear involves tearing of the perineal muscles and the muscles that surround the anus, including the anal sphincter. Third-degree perineal tears also require stitches. Even after repair, long-term consequences of third-degree perineal tears include fecal incontinence, urinary incontinence, and painful intercourse.

FIG. 1D illustrates a fourth-degree perineal tear. Fourth-degree perineal tears are the most severe and involve complete tearing of perineal tissue and underlying muscles between the vaginal opening and the anus. Fourth-degree perineal tears include tearing of the perineal muscles, the anal sphincter, and the tissue lining the rectum. Significant stitching and potentially multiple surgeries may be required to repair fourth-degree tears. Long term consequences of this type of tear also include fecal incontinence, urinary incontinence, and painful intercourse.

During certain stages of labor, including later stages when cervical dilation occurs and pushing begins, including crowning, the perineal tissue stretches. If stretching of the perineal tissue occurs too rapidly or the stress causes by stretching is too large, the perineal tissue tears to varying degrees as described above. However, application of heat to the perineal tissue during these later stages of labor and delivery can reduce the risk of tearing.

Applying the right amount of heat to the perineal tissue at the right time requires a delicate balance. On one hand, enough heat must be applied to the perineal tissue to reduce the risk of tearing. On the other hand, the temperature must not be so great that it irritates, damages, or burns the tissue. In general, the surface temperature of exposed skin in a room-temperature environment can be as low as 31° C. or 32° C. Burn temperature depends on the duration of exposure but generally start at 47° C. or higher. For example, increasing skin tissue temperature, such as perineal body tissue, to 47° C. or higher for extended periods of time, may result in burning or irritation. Temperatures greater than 47° C., such as 48° C. or higher, may be applied to skin without burning if applied for short durations.

As such, embodiments of perineal tissue heating devices described herein are configured to maintaining perineal tissue of a patient between about 32° C. and 47° C. In such embodiments, the perineal tissue of the patient is less prone to perineal tears during labor and delivery. In at least one embodiment, the heating devices described herein maintain perineal tissue between about 35° C. and 45° C., or more preferably between 37° C. and 43° C.

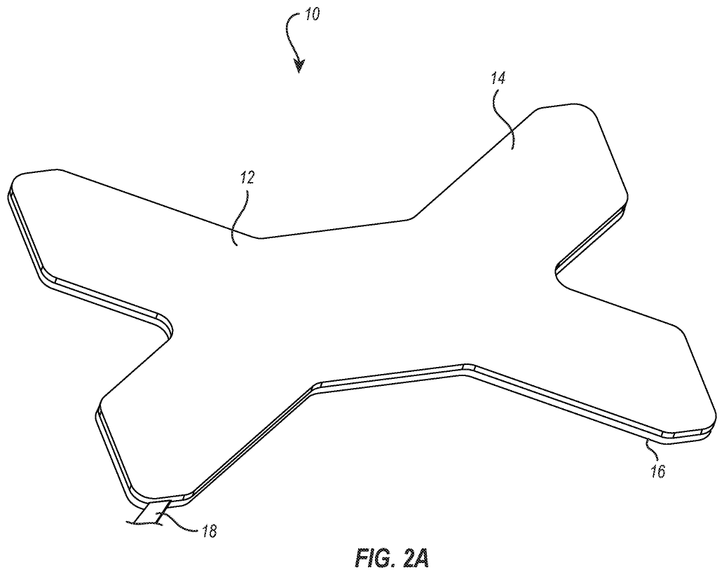

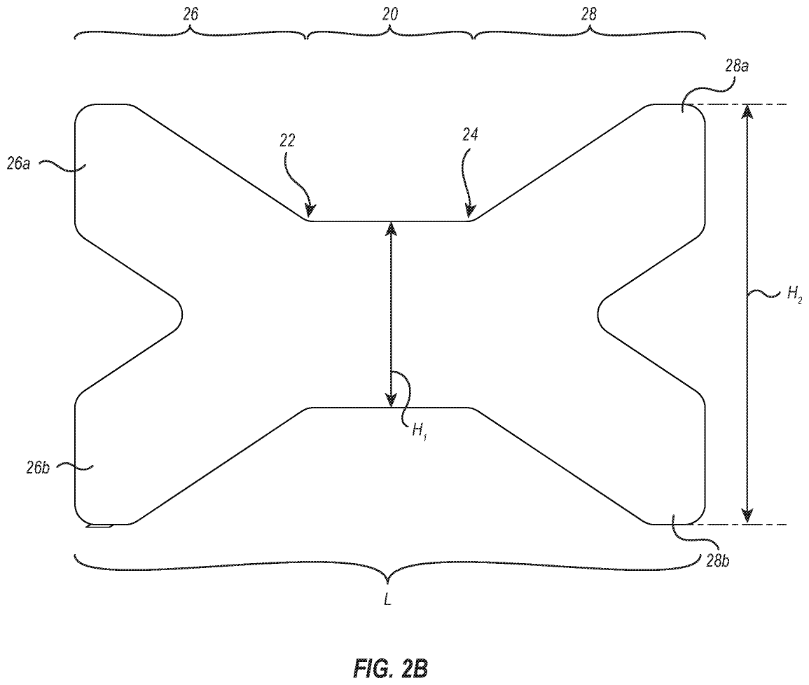

FIG. 2A illustrates a perspective view of a perineal tissue heating device 10, according to an embodiment of the present disclosure. As shown in FIG. 2A, the heating device 10 includes a laminar pad 10 comprising a backing layer 14 and an adhesive layer 16. In addition, in at least one embodiment, the heating device 10 comprises a heating element (not shown in FIG. 2A). In at least one embodiment, the heating element is disposed between the backing layer 14 and the adhesive layer 16 and electrical leads 18 extend out between the layers 14, 16.

FIG. 2B illustrates a top view of the heating device 10 shown in FIG. 2A. The heating device 10 comprises a central portion 20 having a first end 22 and a second end 24. In at least one embodiment, a first end portion 26 extends away from the central portion 20 at the first end 22 and a second end portion 28 extends away from the central portion 20 at the second end 24.

In at least one embodiment of the present disclosure, the central portion 20 may be narrower than the first and second end portions 26, 28 and configured to span the perineal body tissue of a patient between the vaginal opening and anus. For example, the height H₁ of the central portion 20 may be less than the height H₂ of each end portion 26, 28.

For example, in at least one embodiment, the height H₂ of each of the end portions 26, 28 of the heating device 10 is be between about 2 inches and 8 inches, or between about 3 inches and 6 inches, or between about 4 inches and 5 inches. In other embodiments, the height H₂ of each end portion 26, 28 is may be about 6 inches or more, such as about 8 inches, about 10 inches, or greater than 10 inches where circumstances require.

In such embodiments, as noted above, the height H₁ of the central portion 20 is less than the height H₂ of each of the end portions 26, 28. For example, in at least one embodiment, the height H₁ of the central portion 20 is less than or equal to about 2 inches. As such, in at least one embodiment, the height H₁ of the central portion is less than or equal to about 2 inches and the height H₂ of each of the end portions 26, 28 is greater than or equal to about 4 inches. Alternatively, in at least one embodiment, the height H₁ of the central portion is less than or equal to about 2 inches and the height H₂ of each of the end portions 26, 28 is greater than or equal to about 6 inches.

Also, in at least one embodiment, the heating device 10 may have a total length L of about 6-inches. Again, these dimensions may vary in different embodiments. For example, in at least one embodiment, the total length L of the heating device 10 is between about 3 inches and 10 inches. In at least one other embodiment, the total length L of the heating device 10 is between about 4 inches and 8 inches, and preferably between about 5 inches and 7 inches.

Also, the weight of the heating device 10 may vary between embodiments, including between 0.1 lbs. and 0.3 lbs., or between about 0.15 and 0.25 lbs., and preferably less than 0.25 lbs.

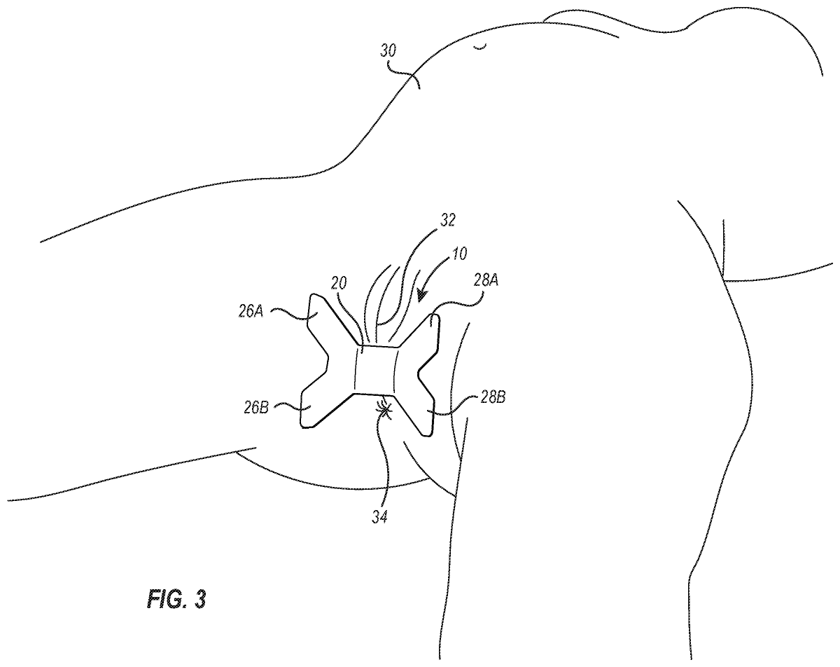

The various dimensions described herein, including the height H₁ of the central portion 20, height H₂ of the end portions 26, 28, and total length L of the heating device 10 are configured to provide ideal geometries for heat application to perineal tissue. For example, the dimensions noted above allow for the central portion 20 to span perineal tissue between the vaginal opening and anus of a patient, without blocking the vaginal opening or anus during use. Also, the end portions 26, 28 are configured to increase surface area available for improved adhesion to tissue and patient anatomy surrounding the perineal tissue. Accordingly, the heating device 10 is sized to be effectively placed over perineal tissue with enough surface area to effectuate adhesion thereto, while being small enough to avoid interference with other medical devices or procedures employed during labor and delivery. More detail regarding the advantageous geometry and placement of the heating device 10 on a patient is given hereafter with reference to FIG. 3.

First, with continued reference to FIG. 2B, in at least one embodiment, the first and second end portions 26, 28 each comprise one or more tabs 26a, 26b and 28a, 28b, respectively. The tabs 26a, 26b of the first end portion 26 extend outward from the first end 22 of the central portion 20. Likewise, the tabs 28a, 28b of the second end portion 28 extend outward from the second end 24 of the central portion 20. Each of the tabs 26a, 26b, 28a, 28b extend at different angles from the central portion 20 and are configured to flex independently of the central portion 20.

As illustrated in FIG. 3, the heating device 10 may be positioned on a patient 30 so that the central portion 20 contacts the perineal tissue between the vaginal opening 32 and anus 34. One will appreciate that the heating device 10 is shaped and dimensioned, as described above, so as to provide heat to the perineal tissue without blocking or otherwise interfering with the vaginal opening 32 or the anus 34. The heating device is also a low-profile, thin laminar pad that conforms to the anatomical contours of the patient. As such, the heating device 10 may be configured to provide pain relief and decreased risk of tearing without interfering with the delivery or postpartum care of the patient.

Advantageously, in at least one embodiment, each tab 26a, 26b, 28a, 28b flexes independently of the central portion 20. As such, the tabs 26a, 26b, 28a, 28b can flex outwardly from the central portion 20 to adhere to the distally extending legs of the patient and other anatomical tissue surrounding the perineum. The tabs 26a, 26b, 28a, 28b flex distally outward and radially around the inside, front, and back of the legs of the patient. In this way, the legs of the patient can move during labor and delivery without affecting the central portion 20 in contact with the perineal tissue. Accordingly, the geometry and dimensions of the tabs 26a, 26b, 28a, 28b shown and described herein provide increased surface area and improved adhesion to the patient while the central portion securely contacts the perineal body tissue of the patient during use.

One will appreciate that the shape, size, and configuration of the heating pad, including the central portion 20, end portions 26, 28 and tabs 26a, 26b, 28a, 28b, may vary without negatively impacting the functionality of the heating device 10, as described herein. For example, in at least one embodiment, the tabs may be circular or triangular in shape, rather than rectangular as shown in FIGS. 2A through 3. Also, for example, in at least one embodiment, the heating device 10 may comprise more or less than two tabs 26a, 26b, 28a, 28b at each end portion 26, 16.

Furthermore, the size, dimensions, and thickness of the heating device 10 may vary. Preferably, the size, dimensions, and thickness of the heating device 10 is such that the heating device 10 spans the perineal tissue between the vaginal opening and anus of the patient to warm the tissue without interfering with the vaginal opening or anus of the patient during labor, delivery, and/or postpartum care.

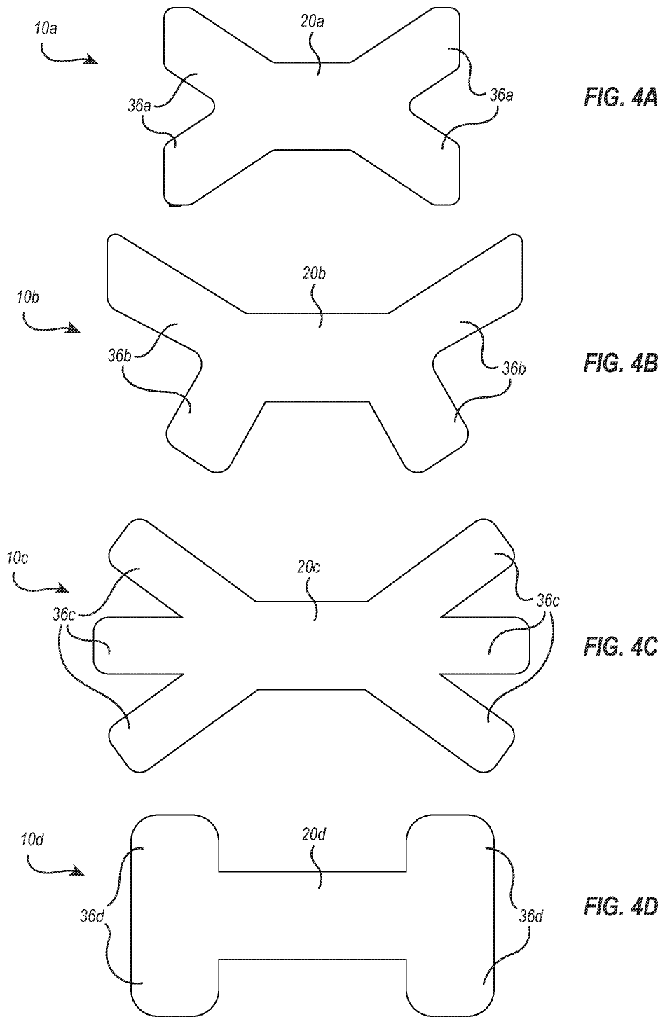

For example, FIGS. 4A through 4D illustrate various embodiments, including different shapes of heating devices 10. FIG. 4A illustrates a heating device 10a that includes two tabs 36a extending from each end of a central portion 20b, similar to the heating device 10 illustrated in the previous figures. Alternatively, as shown in FIG. 4B, the size, shape, and angle at which the tabs 36b extend from the central portion 20b of a heating device 10b may vary. As shown, upper tabs may be longer and narrower than lower tabs and extend at varying angles from the central portion 20b.

As shown in FIG. 4C, at least one embodiment of a heating device 10c includes three tabs 36c extending from each end of the central portion 20c. Increasing the number of tabs 36c may improve adhesion of the heating device 10c to a patient by increasing the surface area that contacts the patient. Alternatively, as shown in FIG. 4D, at least one embodiment of a heating device 10d includes two tabs 36d extending vertically upward and downward from each end of the central portion 20d.

One will appreciate from the foregoing, that any number of shapes and sizes of heating devices 10a-d may be configured, including varying numbers, sizes, and shapes of tabs 36a-d, while accomplishing the advantageous functionalities of the heating device 10 noted above.

As noted above, at least one embodiment of a heating device 10 includes a laminar pad 12 comprising a backing layer 14 and an adhesive layer 16. The backing layer 12 and adhesive layer 16 are secured together to form the laminar pad 10. In at least one embodiment, the backing layer 14 and adhesive layer 16 are adhered together via a separate adhesive or via the adhesive properties of the adhesive layer 16. In at least one embodiment, the backing layer 14 and adhesive layer 16 are integrally formed together, either by molding, thermoforming, or other processes.

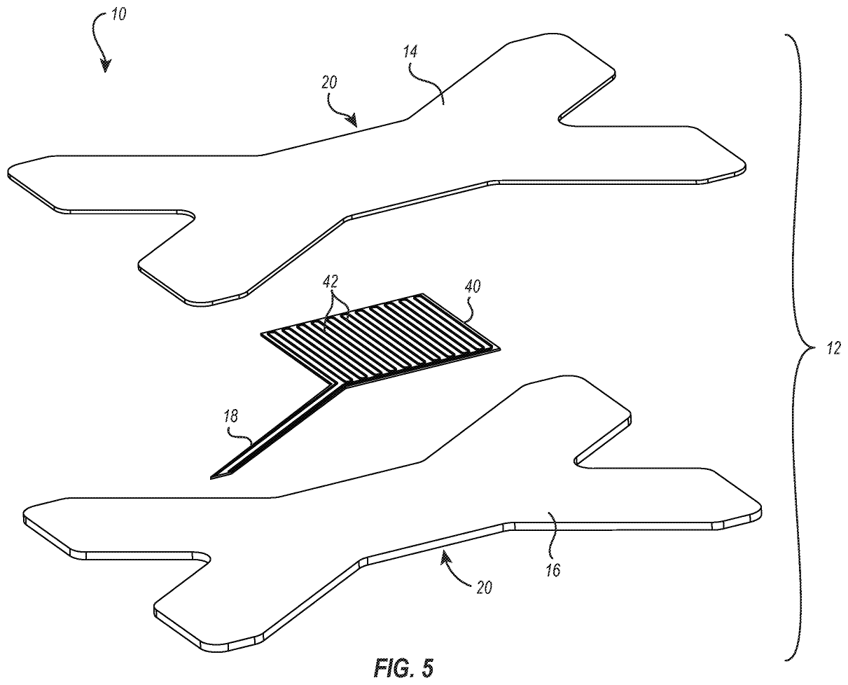

FIG. 5 illustrates an exploded view of an embodiment of a heating device 10 including a laminar pad 12 having a backing layer 14 and an adhesive layer 16. In addition, the heating device 10 comprises a heating element 40. In at least one embodiment, the heating element 40 is disposed between the backing layer 14 and the adhesive layer 16 and electrical leads 18 extend out between the layers 14, 16.

Although the heating element 40 is shown here as an electrical heating element, some embodiments may additionally or alternatively include a chemical heating element. For example, a chemical pack may be provided that is formulated to initiate an exothermic chemical reaction upon activation. In some embodiments, the chemical pack may be a breakable bag, as is known in the art, where puncturing, bursting, or otherwise breaking one part of the pack (e.g., an inner bag) releases a first chemical component so it can contact a second chemical component (e.g., held in an outer bag in which the inner bag was sealed) to allow the exothermic reaction to happen.

In at least one embodiment, the chemical pack may be a sealed bag that when opened, exposes iron or other oxidizable elements to oxygen/air to initiate an exothermic oxidation reaction. In at least one embodiment, the chemical pack may include a sodium acetate solution that crystalizes upon activation, the crystallization resulting in the production of heat. Other heating means known in the art that make use of controlled exothermic reactions may additionally or alternatively be utilized.

The backing layer 14 of the laminar pad 12 may comprise materials that are flexible, soft, tear-resistant, water-proof, and/or biocompatible so as to not irritate the skin of a patient. Such materials may include, but are not limited to, natural materials such as cotton, silk, wool, or other materials such as foams, polyesters, and the like, or combinations thereof. In at least one embodiment, the backing layer 14 comprises a flexible polymer material, including flexible rubbers, plastics, or various other polymer materials. One will appreciate that the backing layer 14 of the heating device 10 may comprise one or a combination of the above noted materials, or other materials generally known or used in the art.

In at least one embodiment of the present disclosure, the adhesive layer 16 of the heating device 10 comprises a layer of non-stick adhesive material disposed on the side of the heating device 10 that contacts the perineal tissue of the patient during use. As used herein, the term “non-stick” or “non-stick adhesive materials” comprise any adhesive material, substrate, or combination of materials and substrates that sufficiently retain the heating device 10 on the tissue during use without leaving residue or tearing hair from the tissue when removed after use. In at least one embodiment, the adhesive layer 16 is formed with the backing layer 14 so that no adhesive application is necessary before placing the heating device 10 on a patient. In at least one embodiment, the adhesive may be separate and applied to the heating device 10 before use or on the perineal tissue.

For example, in at least one embodiment, the adhesive layer 16 may be pre-formed with the heating device 10 before use so that when the heating device 10 is removed from packaging, the adhesive layer 16 is exposed and can be placed on the patient. In yet another embodiment, the heating device 10 may comprise a removable cover layer or film that covers the adhesive layer 16 until use. In such an embodiment, the removable layer may protect the adhesive layer 16 from rubbing off or becoming ineffective before the removable layer is removed prior to use.

As with the materials of the backing layer 14, the non-stick adhesive materials of the adhesive layer 16 preferably comprises materials that do not irritate the skin and can perform properly under wet conditions and a wide range of temperatures, including low and elevated temperatures ranging from between about 10° C. and about 100° C. or higher. In at least one embodiment, for example, the adhesive layer 16 may comprise silicone. In at least one embodiment, the adhesive layer 16 comprises a hydrogel material. In at least one embodiment, the adhesive layer 16 comprises a hydrocolloid material. In at least one embodiment, the adhesive layer 16 comprises one or more polyurethanes or other biocompatible polymer including thermoset or thermoplastic materials. The adhesive layer 16 may also include combinations of the foregoing.

Additionally, or alternatively, the adhesive layer 16 includes a substrate layer, as illustrated in FIG. 5, with an adhesive applied thereon for adhering to the perineal tissue of a patient. As used herein, the term “non-stick adhesive layer” or “adhesive layer” may refer to any of the adhesive layers 16 described herein.

In any of the embodiments described herein, the materials of the backing layer 14 and adhesive layer 16 are preferably configured such that they may be sterilized before use, durable, and water resistant so that the heating device 10 still functions properly in wet conditions. Wet conditions, due to the presence blood, sweat, amniotic fluid, and other fluids near the perineum of a patient during labor and delivery, may vary from 0% humidity to 100% humidity. Also, the materials are preferably such that the heating device 10 performs properly in environments between about 10° C. and about 45° C.

In the illustrated embodiment, the adhesive layer 16 extends across the entirety of the backing layer 14 to ensure that the entire surface area of the laminar pad 12 is adhered to the patient during use. During use, the laminar pad 12 is placed on the patient so that the adhesive layer 16 contacts the tissue of the patient and the backing layer 14 faces distally outward from the patient.

The size, placement, and extent to which the adhesive layer 16 is disposed across the backing layer 14 may vary in one or more embodiments. For example, in at least one embodiment, the adhesive layer 16 may form a layer covering an entire side of the backing layer 14. In at least one embodiment, the adhesive layer 16 may only cover the first and second end portions 26, 28 of the heating device 10, or only the tabs 26a, 26b, 28a, 28b of the heating device 10. In at least one embodiment, adhesive rows may be included along the central portion 20 with or without separate adhesive portions on the tabs 26a, 26b, 28a, 28b.

Also, as seen in the exploded view of FIG. 5, the heating device 10 may comprise a heating element 40 disposed within the central portion 20 of the laminar pad 12 between the backing layer 14 and the adhesive layer 16. The heating element 40 may be formed with either layer 14, 16 or held between layers 14, 16 with the two layers 14, 16 being secured together at least around the perimeter of the heating element 40.

Alternatively, in at least one embodiment, the heating element 20 may be disposed on either side of the heating device 10 or within the material and thickness of either the backing layer 14 or the adhesive layer 16.

In at least one embodiment, the heating element 40 includes a self-limiting heater. Such a self-limiting heating element 40 may comprise, for example, a carbon-silicone matrix material 42 including a thin layer of silicone with conductive carbon particles distributed throughout. When a power source, such as a battery, is connected to the electrical leads 18, the silicone matrix material 42 of the self-limiting thin film heating element 40 expands and increases a distance between each of the carbon particles, thus increasing the electrical resistivity of the material 42. Such a heating element 40 is self-limiting in the sense that no temperature control circuitry or equipment is necessary to maintain the heating element 40 at a desired temperature.

For example, the voltage of a battery connected to the self-limiting heating element 40 determines the temperature reached by the heating element 40. The heating element 40 will not increase in temperature beyond that temperature unless an increased voltage is applied, and vise versa. As such, the heating element 40 advantageously reaches a certain temperature and substantially maintains that temperature, which is based on the voltage of the battery, without the need for other temperature control equipment or feedback loop algorithms.

The absence of external controller equipment, such as control boxes and wires connecting to the heating element 40, results in a reliable, accurate, self-contained, inexpensive, and low maintenance temperature control system built into the heating element 40. Such a system is seamlessly integrated into the hospital setting so as not to interfere with other medical devices or procedures employed during labor and delivery.

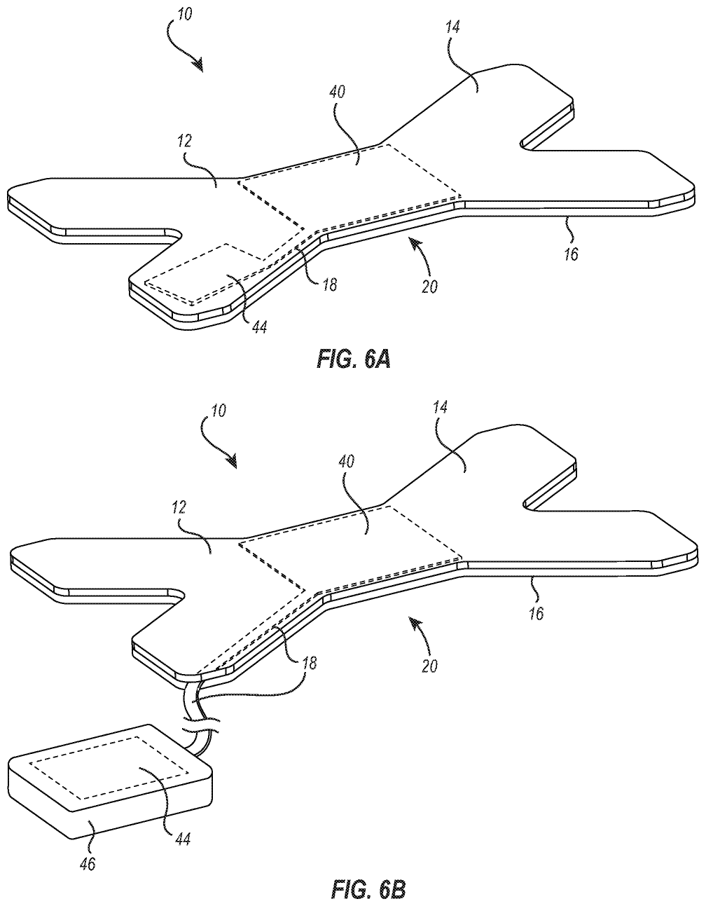

For example, in at least one embodiment shown in FIG. 6A, a battery 44 may be disposed between the backing layer 14 and adhesive layer 16. The battery 44 may be connected to the heating element 40 via electrical leads 18. In at least one embodiment, the battery 44 is disposed on top of the backing layer 14 or separate from the laminar pad 12 entirely.

In another embodiment, as illustrated in FIG. 6B, the self-limiting heating element 40 is powered by a battery 44 disposed outside of the laminar pad 12. In such an embodiment, the electrical leads 18 extend out from between the backing layer 14 and adhesive layer 16 to connect to the external battery 44. Additionally, or alternatively, in at least one embodiment, the battery 44 is housed separately in a housing 46 which may also be adhered to the patient during use. In such an embodiment, the housing is separate from the laminar pad 12 of the heating element 10 and may be adhered to the leg or other tissue surrounding the perineal tissue of the patient during labor and delivery.

One will appreciate that the size, shape, and location of the battery 44 may vary in other embodiments while connecting to the heating element 40 via electrical leads 18.

Alternatively, referring back to FIG. 5, in at least one embodiment, the heating element is a flexible, resistive heating element (or other suitable resistive element known in the art) disposed on or within a soft polymer film. Resistive materials that may be used in embodiments of the heating element 40 include, but are not limited to, metallic alloys such as Nickel alloys, molybdenum alloys, and tungsten alloys, and/or ceramic materials such as graphite or silicon carbide, and/or ceramic metals, or other common resistive materials known in the art.

For example, in at least one embodiment, the heating element comprises etched resistive windings 42 disposed in or on a polymer backing material. In at least one embodiment, the resistive element may include silver or gold. Also, in at least one embodiment, the heating element 40 may include one or more other types of heaters, such as, but not limited to microwave, laser, ferromagnetic, electromagnetic, and ultrasonic heating elements. Other types of heating elements 40 may include positive temperature coefficient (PTC) heating elements, which may comprise one or more ceramic materials.

In at least one embodiment, the heating element 20 comprises an electrically conductive material that is heated through resistive heating when supplied with an electric current. As illustrated in FIG. 5, the heating element 20 may be shaped such that it extends through at least a substantial portion of the central portion 20 of the to laminar pad 12. For example, as shown in FIG. 5, the heating element 20 is configured to zig-zag or snake throughout the central portion 20 of the heating device 10.

The shape and placement of the heating element 40 on or within the heating device 10 may vary. For example, in at least one embodiment, the heating element may be configured in a spiral pattern. Also, for example, at least one embodiment of a heating device 10 may include two or more heating elements 40 disposed on an outside surface and/or within a material thickness of the layers 14, 16 of the laminar pad 12. In any of the embodiments described herein, the heating element 40 is preferably arranged to distribute heat evenly at least throughout the material of the central portion 20 of the heating device 10 that spans and contacts the perineal body tissue of the patient during use.

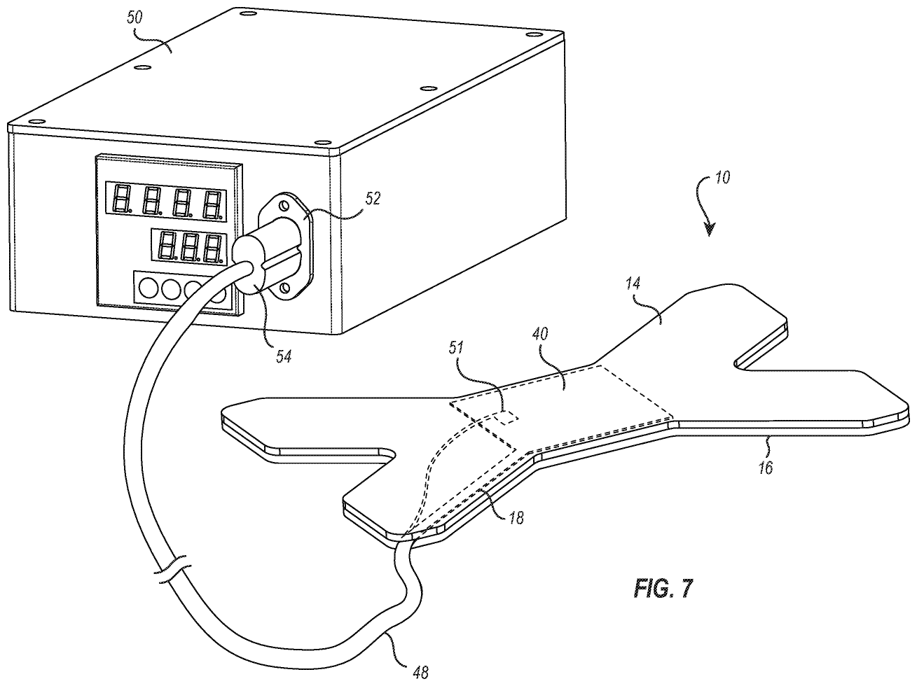

As shown in FIG. 7, the heating element 40 of the heating device 10 may also comprise one or more electrical leads 18 connected to electrical wire 48 that communicates with a power supply from a controller 50. In at least one embodiment, the controller 50 is a digital controller. In at least one embodiment, the controller 50 comprises an analog control circuit with comparators. Furthermore, the controller 50 may be an external controller as shown in FIG. 7, or part of an external battery pack as shown in FIG. 6B. In at least one embodiment, the controller 50 includes an internal battery pack or power supply, as shown in FIG. 6A.

For example, as shown in FIG. 7, in at least one embodiment, a heating element 40 is connected to a controller 50 via one or more wires 48. The controller 50 may be a digital controller that receives feedback data from a temperature sensor 51 disposed in or on the heating device 10. For example, in at least one embodiment, the temperature sensor 51 is disposed between the backing layer 14 and adhesive layer 16 and at or near the heating element 40. Alternatively, or additionally, in at least one embodiment, the temperature sensor 51 is disposed beneath the adhesive layer 16 to directly contact the perineal tissue during use. In at least one embodiment, more than one temperature sensor 51 is disposed at varying locations throughout the heating device 10 as described above.

In at least one embodiment, the controller 50 may vary a supply of electric current to the heating element 40 using a control loop that includes the feedback temperature data from the sensor in order to maintain the temperature of the heating element 40 at a desired temperature.

In at least one embodiment, the controller 50 includes an outlet 52 into which a plug 54 at one end of the wire 48 may be inserted. Alternatively, or additionally, the heating device 10 may have a plug into which the wire 48 can be inserted to supply electric current from the controller 50 to the heating element 40. Furthermore, in at least one embodiment, the wire 48 may be permanently secured to either the controller or the heating pad.

In at least one embodiment where the wires 48 are permanently secured to the heating element 40 of the heating device 10, as discussed above, the other end of the wire 48 may be configured to plug in to the controller 50 so that the wires 48 may be disposed of along with the heating device 10 after use. In still another embodiment, the wires 48 may clip or snap onto the heating device 10 having similar structure to monitoring pads used elsewhere on the patient.

In any of the embodiments of heating elements 40 described herein, the heating element 40 is preferably comprised of flexible, durable materials suited for application to a patient in a hospital setting. For example, the heating element 40 is flexible, light-weight, and durable so that the heating element 40 conforms to the contours of the perineal tissue of the patient, along with the central portion 20 of the laminar pad 12, during use.

In addition, the heating elements 40 described in the present disclosure produce heat that transfers through the adhesive layer 16 or other layers separating the heating element 40 from the perineal tissue of the patient during use. A such, in order to maintain the perineal tissue at a desired temperature, the heating element may need to produce added heat at higher temperatures than the desired temperature of the tissue to compensate for heat dissipation through the adhesive layer 16 or other backing layer 14 of the laminar pad 12.

In any case, embodiments of the heating devices 10 described herein, including embodiments of the heating elements 40 described herein are capable of maintaining the perineal tissue of a patient at a desired temperature of between about 32° C. and 47° C. In such embodiments, the perineal tissue of the patient is less prone to perineal tears during labor and delivery. In at least one embodiment, the heating devices described herein maintain perineal tissue between about 35° C. and 45° C., or preferably between 37° C. and 43° C.

In any case, in the embodiments of heating elements 40 described herein, the controller 50 or battery 44 may raise the actual temperature of the heating element 40 to a desired temperature in less than about 5-minutes and maintain the actual temperature of the perineal tissue of a patient within about 1 to 3° C. of the desired temperature.

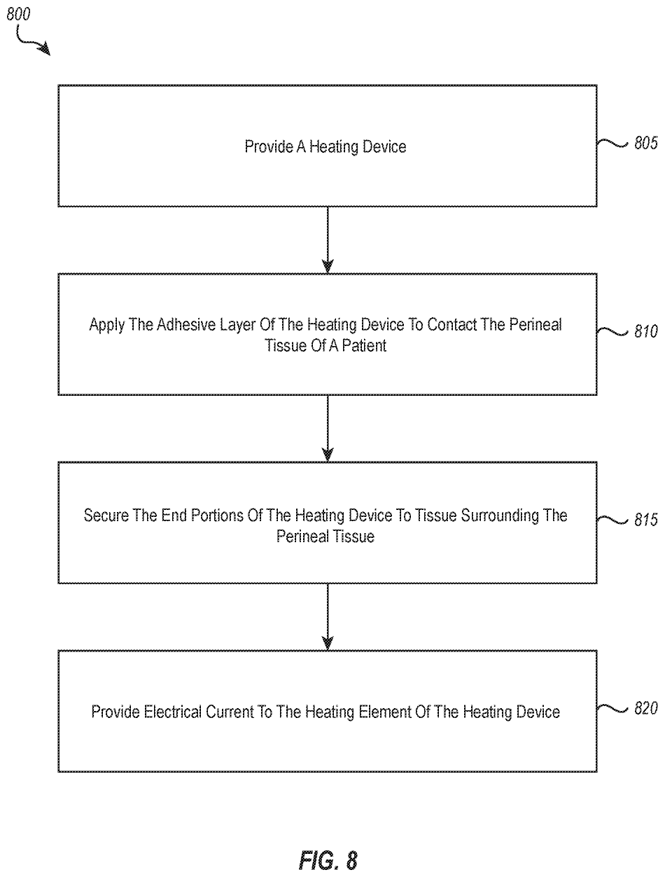

In view of the foregoing, FIG. 8 illustrates a flowchart showing various steps in a method 800 for heating perineal body tissue of a patient during labor and delivery. The method 800 may utilize the various embodiments of a heating device 10 described herein. In a first step 805, a heating device is provided. The heating device may be similar to embodiments of heating devices 10 described herein, which include: a central portion disposed between two opposing end portions; a non-stick adhesive layer; a water-proof backing layer secured to the adhesive layer; and a heating element disposed between the adhesive layer and the backing layer in the central portion.

In at least one embodiment, the method 800 also includes a step 810 comprising applying the non-stick adhesive layer to contact the perineal tissue of a patient. As noted above, this step 810 may optionally comprise the step of removing a removable layer that covers an adhesive that is pre-formed with the heating pad. Next, the method 800 includes the step 815 of securing at least the end portions to tissue surrounding the perineal tissue.

In at least one embodiment, this may be done by pressing the non-stick adhesive layer of the heating pad against the tissue, with the adhesive layer making contact with the patient. Additionally, or alternatively, the method 800 may include a step of securing the central portion to the perineal tissue of the patient via the non-stick adhesive layer.

The method 800 may further include the step 820 of providing electric current to the heating element. As noted above, this may be done by connecting a battery 44 to a self-limiting heating pad 40 and/or connecting a controller 50 to electrical leads 18 of a heating element 40.

In at least one embodiment, the method 800 includes heating the heating element so that the perineal tissue of the patient is warmed to a desired temperature between about 35° C. and 45° C.

In at least one embodiment, the method 800 includes heating the heating element so that the perineal tissue of the patient is warmed to a desired temperature between about 37° C. and 43° C.

In at least one embodiment, the method 800 may include steps of varying the temperature of the heating element so as to vary the temperature of the perineal tissue of a patient during labor and delivery. The temperature variations and gradients of the perineal tissue may be thus controlled to react to conditions such as humidity and ambient temperature or the degree of tissue stretching. The degree of tissue stretching may be detected separately and the information fed into a temperature control feedback loop of a controller, as discussed above. As such, the temperature of the heating element may react to increase temperatures during times of increased stretching and decrease temperatures during times of decreased stretching.

Increased stretching may occur, for example, in the very last stages of labor and delivery when the cervix is fully dilated and the baby is crowning. The perineal tissue of the patient may by stretched the most during this pushing stage of labor. In addition, the stretching of the perineal tissue during these later stages may vary as contractions come and go and the mother pushes intermittently with contractions.

Accordingly, in at least one embodiment of the methods 800 described herein, the heating element may be controlled to increase temperature along with stretching and contractions and/or pushing to further alleviate pain and risk of tearing. Likewise, in at least one embodiment of the method 800, the heating element may decrease in temperature during times of little stretching, such as during early stages of labor and delivery. In this way, the heating device can conserve power and energy for times requiring high temperatures and more power output form the battery or controller.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A device for heating perineal body tissue during vaginal labor and delivery, the device comprising:
a laminar pad comprising:
a backing layer; and
an adhesive layer;
a central portion comprising a heating element;
a first end portion extending from a first end of the central portion; and
a second end portion extending from a second end of the central portion,
wherein the central portion is narrower than each of the first end portion and the second end portion.

2. The device of claim 1, wherein:
the first end portion comprises first and second tabs extending outward from the central portion; and
the second end portion comprises first and second tabs extending outward from the central portion,
wherein the first and second tabs of the first end portion and the first and second tabs of the second end portion are configured to flex independently of the central portion.

3. The device of claim 1, wherein the backing layer comprises a water-proof polymer material.

4. The device of claim 1, wherein the adhesive layer comprises a non-stick adhesive material including at least one of a silicone material and a polyurethane material.

5. The device of claim 1, wherein the heating element is disposed between the backing layer and the adhesive layer.

6. The device of claim 1, wherein the heating element comprises a self-limiting heating element.

7. The device of claim 6, further comprising a battery connected to the self-limiting heating element, the battery configured to provide electrical current to the self-limiting heating element.

8. The device of claim 1, wherein the central portion is configured to:
be applied to perineal tissue between an anus and vaginal opening of a patient during use;
maintain the perineal tissue of a patient between 37° C. and 43° C. during use; and
reduce a risk of perineal lacerations forming during labor and delivery of a patient during use.

9. The device of claim 1, wherein the heating element comprises a resistive heating element on or within a thin polymer film.

10. The device of claim 9, the heating element comprising two or more electrical leads extending from the resistive heating element, the electrical leads being connected to a controller configured to regulate the temperature of the heating element during use.

11. The device of claim 10, further comprising a temperature sensor disposed in or on the central portion of the device, the temperature sensor configured to provide temperature feedback information to the controller.

12. The device of claim 1, wherein the heating element comprises a chemical pack configured to initiate an exothermic reaction upon activation.

13. The device of claim 12, wherein the chemical pack is configured to be activated by breaking a portion of the chemical pack to allow a first component of the chemical pack to contact a second component of the chemical pack.

14. A system for heating perineal body tissue, the system comprising:
a heating pad comprising:
a water-proof backing layer;
a non-stick adhesive layer secured to the backing layer; and
a heating element disposed between the backing layer and the adhesive layer; and
a central portion having a first height, the central portion comprising the heating element; and
a battery connected to the heating element, the battery providing electrical current to the heating element to heat the heating element through resistive heating.

15. The system of claim 14, wherein the heating pad further comprises:
a first end portion extending from the central portion, the first end portion having a second height that is greater than the first height; and
a second end portion extending from the central portion, the second end portion having a third height that is greater than the first height.

16. The system of claim 15, wherein:
the first height is less than or equal to two-inches; and
the second height and the third height are each greater than or equal to four-inches.

17. The system of claim 15, wherein:
the first height is less than or equal to two-inches; and
the second height and the third height are each greater than or equal to six-inches.

18. The system of claim 15, wherein:
the first end portion comprises a first tab and a second tab; and
the second end portion comprises a first tab and a second tab, and
wherein the first and second tab of the first end portion and the first and second tab of the second end portion are configured to flex independently of the central portion when applied to a patient during use.

19. The system of claim 18, wherein the first and second tab of the first end portion and the first and second tab of the second end portion are configured to extend distally along the legs of a patient while the central portion extends across the perineal tissue between the anus and vaginal opening of a patient during use.

20. A method of heating perineal body tissue of a patient during labor and delivery, the method comprising:
providing a heating device, comprising:
a central portion disposed between two opposing end portions; and
a heating element disposed within the central portion;
applying the adhesive layer to contact the perineal tissue of a patient;
securing the end portions to tissue surrounding the perineal tissue; and
activating the heating element.

21. The method of claim 20, wherein activating the heating element comprises providing electrical current to the heating element.

22. The method of claim 20, wherein activating the heating element causes a temperature of the heating element to increase and warm the perineal tissue to a temperature between 35° C. and 47° C.

23. The method of claim 20, wherein activating the heating element causes a temperature of the heating element to increase and warm the perineal tissue to a temperature between 37° C. and 45° C.
